(19) United States
(12) Patent Application Publication
(10) Pub. No.: US 2011/0171212 A1
(43) Pub. Date: Jul. 14, 2011

(54) METHODS AND COMPOSITIONS FOR PREVENTING RADIATION-INDUCED PNEUMONITIS

(75) Inventors: Claus Belka, Tubingen (DE); Jörg Herbst, Heidelberg (DE)

(73) Assignee: APOGENIX GmbH, Heidelberg (DE)

(21) Appl. No.: 12/943,692

(22) Filed: Nov. 10, 2010

Related U.S. Application Data
(63) Continuation of application No. 12/205,250, filed on Sep. 5, 2008, now abandoned.
(60) Provisional application No. 60/970,336, filed on Sep. 6, 2007.

(51) Int. Cl. A61K 39/395; A61K 38/17; A61P 29/00; A61P 11/00; G01N 33/53

(52) U.S. Cl. 424/133.1; 514/21.2; 424/172.1; 436/501; 435/7.1

(57) ABSTRACT

Disclosed are methods of minimizing the risk for a patient of developing pneumonitis during radiotherapy for a thorax-associated neoplasm and compositions for use in such methods. A preferred composition comprises a CD95/CD95L inhibitor. Further disclosed is a method of increasing the radiation dose administered to a patient during radiotherapy for a thorax-associated neoplasm.

### CROSS-REFERENCES TO RELATED APPLICATIONS

This is a continuation of U.S. patent application Ser. No. 12/205,250, entitled “Methods And Compositions For Preventing Radiation-Induced Pneumonitis,” filed Sep. 5, 2008, which claims benefit of U.S. provisional application Ser. No. 60/970,336, entitled “Methods And Compounds For Preventing Radiation-Induced Pneumonitis,” filed Sep. 6, 2007, the disclosure of which is incorporated herein in its entirety by reference.

### FIELD OF THE INVENTION

The present invention refers to a method for treating or preventing radiation-induced pneumonitis in a patient by inhibiting the CD95/CD95L signaling system.

### BACKGROUND OF THE INVENTION

Irradiation-induced pneumonitis is an interstitial pulmonary inflammation that can develop in a considerable proportion of patients which were irradiated because of a thorax-associated neoplasm such as lung cancer, breast cancer, lymphoma, or thymoma. This type of pneumonitis occurs within 1-6 months following radiation treatment, is clinically associated with low-grade fever, cough, and fullness in the chest, and results in a mortality rate of up to 10% (Movsas et al., Chest 1997; 111:1061-76; Roach et al., J Clin Oncol 1995; 13:2606-12; Girinsky et al., J Clin Oncol 2000; 18:981-6). Severe reactions can result in dyspnea, pleuritic chest pain, hemoptysis, acute respiratory distress, and death. Fibrosis can occur without previous pneumonitis but once pneumonitis occurs, fibrosis is almost certain to take place. The radiographic hallmark of irradiation-induced pneumonitis is a diffuse infiltrate corresponding to a previous radiation treatment field. As a consequence, pneumonitis is a dose-limiting side effect of total body irradiation and is the main reason for dose restrictions during radiotherapy for the neoplasms described supra. The rates of pneumonitis rise with increasing radiation dose and irradiated lung volume. Thus, treatment strategies are mainly exerted not to exceed critical normal limits. When limiting the mean lung dose to 18-20 Gy still around 20% of the patients will face a clinically relevant pneumonitic reaction. The strict adherence to normal tissue dose restrictions limits the radiation dose to the tumor, and in turn reduces the probability to control the disease.

Currently, the mechanisms for irradiation-induced pneumonitis are still unclear. Although pneumonitis mostly occurs within the irradiated areas of the lung, it may spread to non irradiated areas, indicating that humoral factors may be involved (Roberts et al., Ann Intern Med 1993; 118:696-700.). Classical irradiation-induced pneumonitis involves direct toxic injury to endothelial and epithelial cells from the radiation, resulting initially in an acute alveolitis. This process leads to an accumulation of inflammatory and immune effector cells within the alveolar walls and spaces. Sporadic radiation pneumonitis, results in an “out-of-field” response. This is thought to be an immunologically mediated process resulting in bilateral lymphocytic alveolitis. Permanent changes of radiation fibrosis can take months to years to evolve but normally stabilize within 1-2 years. Pulmonary fibrosis is the repair process that follows the acute inflammatory response and is characterized by progressive fibrosis of the alveolar septa thickened by bundles of elastic fibers. The process is believed to be a function of activation on cells to produce cytokines and growth factors, which orchestrate most aspects of the inflammatory response.

The current working hypothesis suggests that complex alterations engaging lung epithelial cells (e.g., type 2 pneumocytes) (Penney et al., Int J Radiat Oncol Biol Phys 1994; 29:789-804.), endothelial cells (Hallahan et al., Proc Natl Acad Sci USA 1997; 94:6432-7), and a perpetual cascade of cytokine expression patterns (Rubin et al., Int J Radiat Oncol Biol Phys 1995; 33:99-109; Rube et al., Int J Radiat Oncol Biol Phys 2005; 61:1482-92; Chiang et al., Int J Radiat Oncol Biol Phys 2005; 62:862-71) are important for the induction of pneumonitis (Trott et al., Int J Radiat Oncol Biol Phys 2004; 58:463-9).

CD95 (also known as Fas) and CD95-ligand (CD95-L, also known as FasL or CD178) are expressed in various cells and tissues, including the lung (e.g., bronchiolar and alveolar cells) (Hamann et al., Am J Respir Cell Mol Biol 1998; 19:537-42). CD95 and CD95L are involved in the induction of apoptosis (Krammer, Nature 2000; 407: 789-95; Nagata and Golstein, Science 1995; 267:1449-56), proinflammatory cytokine responses (e.g., tumor necrosis factor [TNF]-α, interleukin [IL]-8) (Hagimoto et al., Am J Respir Cell Mol Biol 1999; 21:436-45; Park et al., J Immunol 2003; 170:6209-16), and the attraction of neutrophils (Seino et al., J Immunol 1998; 161:4484-8; Ottonello et al., J Immunol 1999; 162:3601-6). In this regard, it has been reported that acute lung injury induced by bacterial infection (Grassme et al., Science 2000; 290:527-30), bleomycin-treatment (Kuwano et al., J Clin Invest 1999; 104:13-9), or intrapulmonary deposition of IgG immune complexes (Neff et al., Am J Pathol 2005; 166:685-94) may result in increased CD95 and CD95L expression and the induction of apoptosis and inflammatory responses, including secretion of defensins and/or cytokines. Moreover, the expression of CD95 and CD95L is increased after irradiation (Belka et al., Radiat Res 1998; 149:588-95; Nishioka et al., Int J Mol Med 1999; 3: 275-8.).

To date, there is no treatment method available to efficiently prevent the development of irradiation-induced pneumonitis. In mice, prophylactically administered corticosteroids have been reported to decrease the physiologic effects of radiation. However, in human studies, this approach has failed to prevent the development of clinical pneumonitis. TGF-β has been the target of more recent studies. Angiotensin-converting enzyme (ACE) inhibitors have been reported to decrease the expression of TGF-β1 in animals and recently have been tested in human trials.

Recently, the records of 213 patients receiving thoracic irradiation for lung cancer with curative intent were reviewed. Of these patients, 12% did receive ACE inhibitors for hypertension. Initial results revealed that at the dose used for the treatment of hypertension, ACE inhibitors had no protective effect. Antibiotics and anticoagulants have also been evaluated as treatment options but neither has been found to be clinically beneficial. Pentoxifylline has been reported to decrease late effects from radiation damage but clinical trials in humans have shown no benefit.

Furthermore, U.S. Pat. No. 6,905,684, together with Kuwano et al. (J Clin Invest 1999; 104:13-9) report preventives and remedies for diffuse lung disease. In particular, they report that compounds which inhibit CD95/CD95L system are administered to a patient for treating diffuses lung diseases, preferably cryptogenic interstitial pneumonia and pulmonary fibrosis. However, these references do not provide guidance and leave the question open for the skilled artisan whether the disclosed CD95/CD95L inhibitor is capable of preventing or overcoming the above-described disease known as irradiation-induced pneumonitis or minimizing the risk of developing the same. Consequently, there is still a need for an efficient therapy for the treatment of irradiation-induced pneumonitis.

The inventors of the present invention have now surprisingly found that mice being deficient in expressing either CD95 receptor or its ligand CD95L will not develop radiation-induced pneumonitis as compared to wild type animals.

### BRIEF SUMMARY OF THE INVENTION

In one aspect the present invention provides a method of minimizing the risk for a patient of developing pneumonitis during radiotherapy for a thorax-associated neoplasm. In a preferred embodiment, this method comprises the step of administering to the patient a therapeutically effective amount of a compound which inhibits the CD95/CD95L system in a cell of the patient.

In another aspect of the present invention, a method of increasing the radiation dose administered to a patient during radiotherapy for a thorax-associated neoplasm is provided. In a preferred embodiment, this method comprises the step of administering to the patient a therapeutically effective amount of a compound which inhibits the CD95/CD95L system in a cell of the patient.

Preferably, by using a method of the present invention, the radiation dose administered to a patient during radiotherapy for a thorax-associated neoplasm can be increased by at least 10%, preferably by at least 15%. In some embodiments, the radiation dose administered to a patient during radiotherapy for a thorax-associated neoplasm can be increased by at least 10-15%.

A preferred compound for use in the methods of the present invention is a fusion protein comprising the extracellular domain of CD95. In some embodiments this fusion protein further comprises an oligomerization domain. In other embodiments, this fusion protein further comprises an Fc fragment of an IgG immunoglobulin.

A preferred fusion protein comprises the amino acid sequence shown in SEQ ID NO:1. Another preferred fusion protein consists of the amino acid sequence shown in SEQ ID NO:1. Other preferred fusion proteins for use in the methods of the present invention comprise an amino acid sequence which is at least 90% identical, preferably at least 95% identical to the amino acid sequence of SEQ ID NO:1.

In additional embodiments of the method of the present invention, the compound is an anti CD95 antibody or an anti CD95L antibody.

In yet another aspect of the present invention, a method for identifying a compound for the treatment or prevention of the development of radiation-induced pneumonitis is provided. In a preferred embodiment of the present invention, this method comprises the steps of (a) contacting a peptide derived from the CD95/CD95L system to a test substance under conditions allowing binding of the test substance to the peptide, and (b) determining whether the test substance inhibits an activity of the peptide. Step (b) of this method can be performed by using an in-vitro cell system which is capable of mimicking the cellular conditions occurring with radiation-induced pneumonitis.

Methods, pharmaceutical compositions and kits of the invention embrace the specifics as described herein.

### BRIEF DESCRIPTION OF THE DRAWINGS

FIG. 1 depicts ionizing radiation and alterations of lung physiologic parameters in C57BL6/J wild-type, CD95 receptor-deficient (lpr), and CD95L-deficient (gld) mice. Mice were individually irradiated at the right hemithorax with 0 Gy (sham-irradiated) or 12.5 Gy (irradiated). (A) The breathing frequency ratio (breathing frequency at day x/breathing frequency at day 0) of sham-irradiated and irradiated mice was measured twice weekly from day 0 to 210 and fitted by a non linear regression model that allowed for a sinus function between day 5 and day 70 after irradiation. Regression lines with 95% confidence intervals are shown from day 0 to 90. C57BL6/J mice: sham-irradiated (▴₁, ▴₂, and ▴₃), decreased breathing frequency ratio over time (P<0.001), no pneumonitic peak; irradiated (∘₁, ∘₂, and ∘₃), no change in breathing frequency over time, but a pneumonitic peak was observed (P<0.03). gld mice (GLD in Figure): sham-irradiated (▴₁, ▴₂, and ▴₃), increased breathing frequency ratio over time (P<0.001), no pneumonitic peak observed; irradiated (∘₁, ∘₂, and ∘₃), breathing frequency ratio increased over time (P<0.001), no pneumonitic peak. lpr mice (LPR in Figure): sham-irradiated (▴₁, ▴₂, and ▴₃): increased breathing frequency ratio over time (P<0.001), no pneumonitic peak; irradiated (∘₁, ∘₂, and ∘₃), breathing frequency ratio increased over time (P<0.001), no pneumonitic peak. Details are described in Example 5. (B) Pulmonary resistance (Res) was measured in sham-irradiated mice (bars with ▴) and irradiated mice (bars with ∘) 42 days after irradiation.*; P=0.03, irradiated C57BL6/J mice compared with sham-irradiated control mice. Pulmonary resistance was not altered in lpr (P=0.349) and gld (P=0.349) mice. Details are described in Example 6. (C) Pulmonary compliance (Cdyn) was measured in sham-irradiated mice (bars with ▴) and irradiated mice (bars with ∘) 42 days after irradiation. Pulmonary compliance was not altered in any mouse strain. P=0.14 (C57BL6/J), P=0.37 (lpr), P=0.40 (gld). Each series represents six independent experiments. Pulmonary resistance and pulmonary compliance are expressed in absolute values. Means and upper 95% confidence intervals are shown. The number of independent experiments performed is indicated on each bar. P values (one-sided t-test) were corrected for multiple comparisons according to the false-discovery rate (FDR) procedure using the “R” statistical package. Details are described in Example 6.

FIG. 2 depicts that wild-type mice exhibit clear histopathological alterations of pneumonitis after irradiation, whereas CD95/CD95L deficient mice do not. (A) Images of histological sections. At day 1 and day 21 histological sections were taken from lungs of sham-irradiated and irradiated C57BL6/J, lpr (CD95 deficient), and gld (CD95L deficient) mice, stained with hematoxillin and eosin and analysed at 10× and 40× (inset) magnification. (B) Semi-quantitative histological analysis of pulmonary inflammation. In contrast to CD95/CD95L deficient mice, C57BL6/J mice show a strongly elevated pulmonary inflammation sum score after irradiation. Histological sections were prepared at day 1, 21, 42, and 84 as described in FIG. 2A and semi-quantitatively analyzed to obtain the pulmonary inflammation score (numerical score consisting of alveolar wall thickness, interstitial edema, and interstitial and peribronchial inflammation). C57BL6/J: right and left lung: day 1: n.s.; day 21, day 42, and day 84: P<0.001. gld: right and left lung: day 1: P<0.001 (sum inflammation score significantly lower); day 21, day 42, and day 84: n.s. lpr: right and left lung: day 1, day 21, day 42, and day 84: n.s. (C) Differences in pulmonary inflammation score of irradiated and sham-irradiated littermates. On the basis of the data from FIG. 2B the delta inflammation score values (delta inflammation score=inflammation score [irradiated mice]−inflammation score [sham-irradiated mice]) were calculated and plotted against time. For histopathological analysis at least 5 C57BL6/J, gld and lpr mice per dose and time point were examined. Details are described in Example 7.

### DETAILED DESCRIPTION OF THE INVENTION

Accordingly, the object of the present invention refers to a method of treating or preventing, particularly of minimizing the risk for a patient of developing pneumonitis during radiotherapy for a thorax-associated neoplasm, the method comprising administering to said patient a therapeutically effective amount of a compound which inhibits the CD95/CD95L system.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

It must be noted that, as used in the specification and the appended claims, the singular forms “a,” “an” and “the” include plural referents unless the context clearly dictates otherwise. Thus, for example, reference to “a compound” includes mixtures of compounds, reference to “a pharmaceutical carrier” includes mixtures of two or more such carriers, and the like.

The term “minimizing the risk”, as used herein in relation to the development of pneumonitis, is to be understood as embracing both symptomatic and prophylactic modes, that is the immediate treatment, e.g. of acute pneumonitis (symptomatic treatment), or already manifested pneumonitis upon radiation, as well as advance treatment to prevent, ameliorate or restrict long term symptomatology (prophylactic treatment).

For the avoidance of doubt, as used herein, the term “pneumonitis during radiotherapy” is synonymous to irradiation-induced pneumonitis, radiation-induced pneumonitis or, in the context of the present invention sometimes only to “pneumonitis”. As used herein, the term “pneumonitis” refers to a pulmonary inflammatory disease which is a result of a radiation dose that is sufficient, under given circumstances, to induce pneumonitis in a patient, as further described infra.

The term “radiotherapy”, as used herein, is a synonym for “radiation” or “irradiation” and refers to the use of electromagnetic or particulate radiation in the treatment of a thorax-associated neoplasia. Radiation therapy is based on the principle that high-dose radiation delivered to a target area will result in the death of reproductive cells in both tumor and normal tissues. The radiation dosage regimen absorbed in living tissue is generally defined in terms of “gray” (Gy; 1 Gy=1 joule of radiation energy absorbed per kilogram of tissue), time and fractionation, and must be carefully defined by the oncologist. The amount of radiation a patient receives will depend on various consideration but the two most important considerations are the location of the tumor in relation to other critical structures or organs of the body, and the extent to which the tumor has spread. Examples of radiotherapeutic agents are provided in, but not limited to, radiation therapy and are known in the art (Hellman, Principles of Radiation Therapy, Cancer, in Principles and Practice of Oncology, 24875 (Devita et al., ed., 4th ed., v 1, 1993). Recent advances in radiation therapy include three-dimensional conformal external beam radiation, intensity modulated radiation therapy (IMRT), stereotactic radiosurgery and brachytherapy (interstitial radiation therapy), the latter placing the source of radiation directly into the tumor as implanted “seeds.” These newer treatment modalities deliver greater doses of radiation to the tumor, which accounts for their increased effectiveness when compared to standard external beam radiation therapy. Beta-emitting radionuclides are considered the most useful for radiotherapeutic applications because of the moderate linear energy transfer (LET) of the ionizing particle (electron) and its intermediate range (typically several millimeters in tissue). Gamma rays deliver dosage at lower levels over much greater distances. Alpha particles represent the other extreme; they deliver very high LET dosage, but have an extremely limited range and must, therefore, be in intimate contact with the cells of the tissue to be treated. In addition, alpha emitters are generally heavy metals, which limits the possible chemistry and presents undue hazards from leakage of radionuclide from the area to be treated. Depending on the tumor to be treated all kinds of emitters are conceivable within the scope of the present invention.

The term “thorax-associated neoplasm” encompasses tumors of the upper part of the body, for example neck cancers such as thyroid cancer and esophagus tumor, cancers of the torso such as lung cancer, breast cancer, lymphoma, or thymoma, as well as tumor of the upper gastric system such as stomach cancer, or skin tumors in the thorax area.

For the purposes of convenience, the term “compound which inhibits the CD95/CD95L system” is hereinafter referred to as “CD95/CD95L inhibitor”. The CD95/CD95L inhibitor can be any compound that interferes with the expression and/or the function of the death receptor CD95 or its ligand CD95L. Such compounds and their synthesis are already well-described in the art, for example in WO 2004/071528, WO 01/41803, and WO 95/27735 which are incorporated herein by reference, and include antibodies and fragments thereof, aptamers, siRNA, antisense RNA, muteins, small molecules interfering with the CD95/CD95L interaction and fusion proteins mimicking the structure of either CD95 or CD95L without fulfilling their cellular functions.

Preferably, the CD95/CD95L inhibitor is a fusion protein comprising the extracellular domain of CD95. Fusion proteins comprising the extracellular domain of CD95 which are conceivable within the scope of the present invention are well-described in the patent family of WO 2004/085478 and all members thereof, the content of which being incorporated herein by reference. The fusion protein may also contain domains that enable the oligomerization, preferably trimerization of said fusion protein. Most preferably, the fusion protein comprises the sequence of SEQ ID NO:1, a fragment or derivative thereof.

As used herein, the term “fragment or derivative” means that this amino acid sequence differs from that of SEQ ID NO:1 in one or more positions and display a high degree of homology to said sequence. Homology means a sequence identity over the entire length of the amino acid sequence shown in SEQ ID NO:1 of at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least greater than 99%. The deviations to SEQ ID NO:1 can derive from deletion, addition, substitution or insertion of amino acids. To determine the percentage sequence identity preferably at least 30%, 40%, 50%, 70%, 80%, 85%, 86%, 87%, 88%, 89%, 90%, 91%, 92%, 93%, 94%, 95%, 96%, 97%, 98%, 99% or more of the length of the reference sequence are compared.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., Altschul et al., Nucl. Acids Res. 25:3389-3402 (1977) and Altschul et al., J. Mol. Biol. 215:403-410 (1990)). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length. Preferably the identity exists over a region comprising amino acid residues 17 to 172 of the amino acid sequence shown in SEQ ID NO:1.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nucl. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “fragment” typically comprises 8, 10, 12, 14, 16 or more neighboring amino acid residues of SEQ ID NO:1. Such fragments can be chosen due to their biological activity or due to a different function, e.g. binding to a specific substrate or effect as immunogenic. Predicted domains or functional regions can be identified by methods known in the art, e.g. computer programs such as PROSITE analysis. “Derivative” can also mean that one or more amino acid is chemically modified or labeled. Such chemical modification can have the effect that the CD95/CD95L inhibitor is stabilized or acquires other desired chemical of physical properties. Such modifications known to the skilled artisan include acetylation, acylation, ADP-ribosylation, amidation, covalent attachment of flavin, nucleotides, lipids, or phosphotidylinositol, crosslinking, formylation, glycosylation, GPI-anchor formation, hydroxylation, methylation, myristoylation, oxidation, proteolytic processing, phosphorylation, prenylation, selenoylation, pegylation, and the like. Such modifications are known to the person skilled in the art and are described in the literature (e.g. Creighton et al., “Proteins-Structure and Molecular Properties”, 2nd Ed., 1993, W. H. Freeman & Company, New York).

As used herein, “therapeutically effective amount” of one of the CD95/CD95L inhibitor means a sufficient amount of said compound to treat a particular disease, at a reasonable benefit/risk ratio. In general, the term “therapeutically effective amount” shall refer to an amount of said compound which is physiologically significant and improves an individual's health. An agent, i.e. said compound, is physiologically significant if its presence results in a change in the physiology of the recipient human. For example, in the treatment of a pathological condition, administration of said compound which relieves or arrests further progress of the condition would be considered both physiologically significant and therapeutically effective. Said compound may be employed in pure form or, where such forms exist, in pharmaceutically acceptable salt, ester or prodrug forms.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977), which is incorporated herein by reference. The salts can be prepared in situ during the final isolation and purification of the CD95/CD95L inhibitors, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, berate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydmxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate. 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl sulfonate and aryl sulfonate.

As described herein, the development of pneumonitis is a dose-limiting side effect of total body irradiation and is the main reason for dose restrictions during radiotherapy for thorax-associated neoplasm. Therefore, it is conceivable within the scope of the present invention to use the CD95/CD95L inhibitor for a method of increasing the radiation dose administered to a patient during radiotherapy for a thorax-associated neoplasm, the method comprising administering to said patient a therapeutically effective amount of a compound which inhibits the CD95/CD95L system.

Methods of determining the required radiation dose are known to the skilled physician. The probability of developing irradiation-induced pneumonitis correlates with the irradiated lung volume. To measure the occurrence of pneumonitis as a response to radiation in patients in the presence or absence of a CD95/CD95L inhibitor, and to determine the dose of radiation that is sufficient to cure the neoplasm but low enough to avoid the development of pneumonitis, clinical and radiological symptoms within 6 months after the end of the radiotherapy may be evaluated. A clinically manifested pneumonitis corresponds to “common toxicity criteria” CTC grade 3-4 or “radiation therapy oncology group” RTOG grade 1-2 and more (for detailed reference, see homepage of RTOG (Radiation Therapy Oncology Group) Foundation, Philadelphia, Pa.). The pulmonary volume can be measured after different three-dimensional models. The lung dose (pulmonary applied dose), the effective lung volume (lung volume irradiated with a timor-effective total dose), as well as volumes irradiated with 10-30 Gray (Gy) can be analyzed via logistic regression. To calculate predictive values for irradiation induced pneumonitis, Receiver Operating Characteristic (ROC), simply “ROC curves”, are generated. A ROC curve is a graphical plot of the sensitivity vs. (1-specificity) for a binary classifier system as its discrimination threshold is varied. The ROC can also be represented equivalently by plotting the fraction of true positives (TPR=true positive rate) vs. the fraction of false positives (FPR=false positive rate). For the purposes of the instant invention, the risk for developing irradiation induced pneumonitis (in %) is plotted against the relative (percentage) moiety of the total lung volume less the volume of the tumor to be irradiated (see for example, Int. J. Radiat. Oncol. Biol. Phys. 2007 Feb. 1; 67(2):410-6). Since the usually tolerated dose for the development of irradiation-induced pneumonitis lies within 25-30 Gy, it is conceivable to anticipate within the scope of the present invention that, in the presence of a therapeutically effective amount of a CD95/CD95L inhibitor, higher radiation doses can be applied, nonetheless depending on age, sex, general physical stage, FEV1 (forced expiratory volume), site of tumor, smoking habits, alteration of TGF-beta and chemotherapies. Preferably, the radiation dose can be increased by at least 3%, at least 5%, at least 8%, at least 10%, at least 15% as compared to the dose typically applied in the absence of the CD95/CD95L inhibitor. Typically, the radiation to be applied can be increased by 10 to 15%.

As described supra, the CD95/CD95L inhibitors are useful in the treatment or prevention of radiation-induced pneumonitis occurring upon irradiation of thorax-associated tumors or as a mean to increase radiation dose during radiotherapy of thorax-associated neoplasm.

For the above purposes, some CD95/CD95L inhibitors preferably will be administered topically within the airways, e.g. by the pulmonary route, by inhalation. While having potent efficacy when administered topically, CD95/CD95L inhibitors are devoid of, or exhibit relatively reduced, systemic activity, e.g. following oral administration. CD95/CD95L inhibitors thus provide a means for the treatment of pneumonitis with the avoidance of unwanted systemic side effect, e.g., consequent to inadvertent swallowing of drug substance during inhalation therapy. (It is estimated that during the course of maneuvers required to effect administration by inhalation, up to 90% or more of total drug substance administered will inadvertently be swallowed rather than inhaled). By the provision of CD95/CD95L inhibitors which are topically active, e.g. effective when inhaled but systemically inactive, the present invention makes CD95/CD95L inhibitor therapy available to subjects for whom such therapy might otherwise be excluded, e.g., due to the risk of systemic, in particular immunosuppressive, side effects.

Accordingly, the present invention further refers to pharmaceutical preparations and compositions for the treatment of radiation-induced pneumonitis which comprises a CD95/CD95L inhibitor and, optionally, a pharmaceutically acceptable carrier, and methods for preparing such phannaceutical compositions. The methods for preparing pharmaceutical compositions, i.e. medicaments, are known per se to the skilled artisan.

The specific therapeutically-effective dose level for any particular patient will depend upon a variety of factors including the severity of the pneumonitis; activity of the specific CD95/CD95L inhibitor employed; the specific composition employed; the age, body weight, general health, sex and diet of the patient; the time of administration, route of administration, and rate of excretion of the specific CD95/CD95L inhibitor employed; the duration of the treatment; drugs used in combination or coincidental with the specific CD95/CD95L inhibitor employed; and like factors well known in the medical arts. For example, it is well known within the skill of the art to start doses of the CD95/CD95L inhibitor at levels lower than required to achieve the desired therapeutic effect and to gradually increase the dosage until the desired effect is achieved.

As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, phosphate buffer solutions; non-toxic, compatible lubricants such as sodium lauryl sulfate and magnesium stearate; as well as coloring agents, releasing agents, coating agents, sweetening, flavoring and perfuming agents. Preservatives and antioxidants can also be present in the composition, according to the judgment of the formulator.

The compositions may be administered to humans and other animals orally, rectally, parenterally, intracisternally, intravaginally, intraperitoneally, topically (as by powders, ointments, drops or transdermal patch), bucally, or as an oral or nasal spray. The term “parenteral” as used herein refers to modes of administration which include intravenous, intramuscular, intraperitoneal, intrasternal, subcutaneous and intraarticular injection and infusion.

Dosage forms for topical or transdermal administration of CD95/CD95L inhibitors include ointments, pastes, creams, lotions, gels, plasters, cataplasms, powders, solutions, sprays, inhalants or patches. The active component, i.e. the CD95/CD95L inhibitor, is admixed under sterile conditions with a pharmaceutically acceptable carrier and any needed preservatives or buffers as may be required. The ointments, pastes, creams and gels may contain, in addition to an active CD95/CD95L inhibitor of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to CD95/CD95L inhibitors, excipients such as lactose, talc, silicic acid, aluminum hydroxide, calcium silicates and polyamide powder, or mixtures of these substances. Sprays can additionally contain customary propellants such as chlorofluorohydrocarbons. For nasal administration, CD95/CD95L inhibitors will suitably be administered in liquid or powdered form from a nasal applicator.

It is clear that safety may be maximized by delivering the drugs by the inhaled route either in nebulizer form or as dry powder. Clearly the great advantage of the inhaled route, over the systemic route, in the treatment of radiation-induced pneumonitis and other diseases of airflow obstruction, is that patients are exposed to very small quantities of the drug and the CD95/CD95L inhibitor is delivered directly to the site of action.

CD95/CD95L inhibitors therefore are preferably employed in any dosage form appropriate for topical administration to the desired site. Thus, for the treatment of pneumonitis, a CD95/CD95L inhibitor may be administered via the pulmonary route/by inhalation from an appropriate dispenser device. For this purpose, a CD95/CD95L inhibitor may be employed in any suitable finely dispersed or finely dispersible form, capable of administration into the airways or lungs, for example in finely divided dry particulate form or in dispersion or solution in any appropriate (i.e., pulmonarily administerable) solid or liquid carrier medium. For administration in dry particulate form, a CD95/CD95L inhibitor may, for example, be employed as such, i.e., in micronised form without any additive materials, in dilution with other appropriate finely divided inert solid carrier or diluent (e.g., glucose, lactose, mannitol, sorbitol, ribose, mannose or xylose), in coated particulate form or in any other appropriate form as known in the art for the pulmonary administration of finely divided solids. Pulmonary administration may be effected using any appropriate system as known in the art for delivering drug substance in dry or liquid form by inhalation, e.g. an atomizer, nebulizer, dry-powder inhaler or like device. Preferably a metered delivery device, i.e., capable of delivering a pre-determined amount of a CD95/CD95L inhibitor at each actuation, will be employed. Such devices are known in the art.

Pharmaceutical compositions of this invention for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include, but are not limited to, water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, an active CD95/CD95L inhibitor is mixed with at least one inert, pharmaceutically-acceptable excipient or carrier, such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules may be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically-acceptable emulsions, solutions, suspensions, syrups and elixirs. In addition to an active CD95/CD95L inhibitor, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Suspensions may contain, in addition to the active CD95/CD95L inhibitor, a suspending agent, for example, ethoxylated isostearyl alcohols, polyoxyethylene sorbitol and sorbitan esters, microciystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, may be prepared as a dry powder which may be pressurized or non-pressurized. In non-pressurized powder compositions, the active ingredient in finely divided form may be used in admixture with a larger-sized pharmaceutically-acceptable inert carrier comprising particles having a size, for example, of up to 100 micrometers in diameter. Suitable inert carriers include sugars such as lactose. Desirably, at least 95% by weight of the particles of the active ingredient have an effective particle size in the range of 0.01 to 10 micrometers. Alternatively, the composition may be pressurized and contain a compressed gas, such as nitrogen or a liquified gas propellant. The liquified propellant medium and indeed the total composition are preferably such that the active ingredient does not dissolve therein to any substantial extent. The pressurized composition may also contain a surface-active agent, such as a liquid or solid non-ionic surface-active agent or may be a solid anionic surface-active agent. It is preferred to use the solid anionic surface-active agent in the form of a sodium salt.

CD95/CD95L inhibitors may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming Liposomes can be used. The present compositions in liposome form can contain, in addition to a CD95/CD95L inhibitor, stabilizers, preservatives, excipients, and the like. The preferred lipids are the phospholipids and the phosphatidyl cholines (lecithins), both natural and synthetic. Methods to form liposomes are known in the art (see, for example, Prescott, Ed., Methods in Cell Biology, Volume XIV, Academic Press, New York, N.Y. (1976), p. 33 et seq.).

Another object of the present invention refers to a method for identifying a compound useable for the treatment or prevention of the development of radiation-induced pneumonitis comprising the steps: (a) contacting of a peptide derived from the CD95/CD95L system to a test substance under conditions allowing the binding of said test substance to said peptide, and (b) determination, whether said test substance inhibits the activity of said peptide. Preferably, the determination of step (b) is performed by using an in-vitro cell system capable of mimicking the cellular conditions occurring with radiation-induced pneumonitis.

For use in diagnostic, research, and therapeutic applications described above, kits are also provided by the present invention.

Reference to particular buffers, media, reagents, cells, culture conditions and the like, or to some subclass of the same, is not intended to be limiting, but should be read to include all such related materials that one of ordinary skill in the art would recognize as being of interest or value in the particular context in which they are presented. For example, it is often possible to substitute one buffer system or culture medium for another, such that a different but known way is used to achieve the same goals as those to which the use of a suggested method, material or composition is directed.

In addition, a kit may include instructional materials containing directions (i.e., protocols) for the practice of the methods of this invention. The instructions may be present in the subject kits in a variety of forms, one or more of which may be present in the kit. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Optionally, the instruction comprises warnings of possible side effects and drug-drug or drug-food interactions.

A wide variety of kits and components can be prepared according to the present invention, depending upon the intended user of the kit and the particular needs of the user. Kit embodiments of the present invention include optional functional components that would allow one of ordinary skill in the art to perform any of the method variations described herein.

Although the forgoing invention has been described in some detail by way of illustration and example for clarity and understanding, it will be readily apparent to one of ordinary skill in the art in light of the teachings of this invention that certain variations, changes, modifications and substitutions of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The referenced patents, patent applications, and scientific literature, including accession numbers to GenBank database sequences, referred to herein are hereby incorporated by reference in their entirety as if each individual publication, patent or patent application were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As can be appreciated from the disclosure above, the present invention has a wide variety of applications. The invention is further illustrated by the following examples, which are only illustrative and are not intended to limit the definition and scope of the invention in any way.

### EXAMPLES

### Example 1

### Animal Husbandry

Four-to-six week-old female C57BL6/J wild-type (n=67), CD95 receptor-deficient (lpr) (n=53), or CD95L-deficient (gld) (n=61) mice (Charles River laboratories, Sulzfeld, Germany) were housed up to five mice per cage in a standard barrier facility at room temperatures of 20-22° C. with a 12-hour light/dark cycle. Food (Laboratory animal diets, 3430.0.15, Provimi Kliba A G, Kliba Nafag, CH-4303 Kaiseraugst, Switzerland; standard laboratory diet, Altromin-1310, Altromin, D-32791 Lage, Germany) and drinking water were provided ad libitum. Mice were subsequently enrolled to the study protocol with a body weight of approximately 20 g after adaptation to a total-body plethysmograph for 14 days. All mouse protocols were approved by the University of Tubingen animal protection board in conjunction with the regional council Tubingen (Regierungsprasidium Tübingen).

Animal care was provided in accordance with the guidelines for care and use of laboratory animals (newest edition 25.05.1998 BGBI. I S. 1105; animal experiment R 3/01; R 1/04). Mice were anesthetized with 2% isoflurane, were placed in holders, and their bodies, excluding the right hemithorax, shielded with 60 mm of lead. Mice were then irradiated with a single dose of 0 Gy (sham) or 12.5 Gy using a linear accelerator (dose rate=4.7 Gy/min; n>5 mice/dose group).

### Example 2

### Measuring Breathing Frequency

After irradiation, breathing frequency was measured using a total-body plethysmograph with a chamber volume of 960 cm³. Mice were placed individually in the chamber, and pressure changes in the chamber were monitored, converted into an electric signal, which was filtered to remove interfering signals not caused by respiration, and amplified. The amplified signal was calibrated from 1 to 8 Hz (i.e., breaths per second) using an oscillator. Breathing frequencies at rest were measured for 2 minutes and 40 seconds at the same time of day to minimize circadian changes. Breathing frequency was measured twice weekly for up to 30 weeks, and the breathing frequency ratio (breathing frequency at day x/breathing frequency at day 0) was plotted as a function of time.

The user defined non linear regression module of a statistical software package (Statistica, StatSoft, Inc. Tulsa, USA, Version 5.5) was used. The following three-parameter model was used to fit the breathing frequency ratio (BR):

The untransformed breathing rate (BRᵤ) as well as an area under the curve transformation of the BRᵤ data and the breathing frequency ratio (BR; BR=BRᵤ at a specific time after radiotherapy/BRᵤ before start of radiotherapy) were basis of the statistical analysis. BRᵤ and BR were normally distributed.

The best fittings were derived by using the following three-parameter model using BR as dependent variable: BR=A*T+C+B*(1+sin(4.683333+((T−5)/10.34653)))*((T>5) and (T<70)). Loss function: (Observed−Predicted)**2.

T=time [days]. A and C are the parameters of simple linear regression. B represents a parameter for an additional sinus function that was allowed between days 5 and 70 to adapt the linear regression for the pneumonitis peak of the BR. The constant parameters 4.683333 and 10.34653 were necessary to set the sinus function to zero at days 5 and 70, respectively. Asymptotic confidence intervals and P values were estimated based on a Hesse matrix. For the breathing frequency analysis, up to 33/34 sham irradiated/irradiated C57BL6/J mice, 32/29 sham irradiated/irradiated gld and 27/26 sham irradiated/irradiated lpr mice were analyzed dependent on the day of analysis (Tables 1-3).

TABLE 1
Number of C57BL6/J Mice for Breathing Frequency Analysis

| Dose | Day 1 | Day 21 | Day 42 | Day 84 | Day 210 |
|---|---|---|---|---|---|
| 0 Gy | 33 | 26 | 19 | 12 | 5 |
| 12.5 Gy | 34 | 27 | 20 | 13 | 6 |

TABLE 2
Number of gld Mice for Breathing Frequency Analysis

| Dose | Day 1 | Day 21 | Day 42 | Day 84 | Day 210 |
|---|---|---|---|---|---|
| 0 Gy | 32 | 27 | 20 | 13 | 6 |
| 12.5 Gy | 29 | 23 | 17 | 12 | 6 |

TABLE 3
Number of lpr Mice for Breathing Frequency Analysis

| Dose | Day 1 | Day 21 | Day 42 | Day 84 | Day 210 |
|---|---|---|---|---|---|
| 0 Gy | 27 | 22 | 17 | 11 | 6 |
| 12.5 Gy | 26 | 21 | 16 | 10 | 5 |

### Example 3

### Measuring Pulmonary Resistance And Pulmonary Compliance

Pulmonary resistance and pulmonary compliance, additional parameters of lung physiology that may be indicative of lung injury, were analyzed in wild-type, gld, and lpr mice at day 42 indicative of the maximum increase of breathing frequency ratio in irradiated C57BL6/J mice. To do this, mice were deeply anesthetized with pentobarbital and their lungs prepared and perfused at the indicated time points after irradiation, as described (Held et al., Br J Pharmacol 1999; 126:1191-9; Held and Uhlig, Am J Respir Grit Care Med 2000; 162:1547-52).

Mouse lungs were prepared and perfused at indicated time point after irradiation essentially as described (Watanabe-Fukunaga et al., Nature 1992; 356:314-7; Takahashi et al., Cell 1994; 76(6):969-76). Briefly, lungs were perfused in a nonrecirculating fashion through the pulmonary artery at a constant flow of 1 ml/min resulting in a pulmonary artery pressure of 2-3 cm H₂O. For perfusion we used RPMI 1640 medium lacking phenol red (Biowhittaker) at 37° C. and containing 4% low endotoxin grade albumin (Serva). The lungs were ventilated by negative pressure (−2 cm H₂O to −10 cm H₂O) at a rate of 90 breaths/min. Artificial thorax chamber pressure was measured with a differential pressure transducer (DP 45-24; Validyne), and the airflow rate was measured with a Fleisch-type pneumotachograph tube connected to a differential pressure transducer (DP 45-15; Validyne). Arterial pressure was continuously monitored by means of a pressure transducer (Isotec Healthdyne) that was connected to the cannula ending in the pulmonary artery. All data were transmitted to a computer and analyzed with Pulmodyn software (Hugo Sachs Elektronik). Tidal volume (V_{T}) was derived by integration of the flow rate, and the data were analyzed by applying the formula: P=1/C·V_{T}+R_{L}·dV_{T}/dt, where P is chamber pressure, C is pulmonary compliance, and R_{L} lung resistance. Data regarding airway resistance and compliance were obtained between 20 and 30 min of perfusion and ventilation. The data were analyzed by one-sided t-tests. The p-values were corrected for multiple comparisons according to the false-discovery rate procedure using the “R” (R: A language and environment for statistical computing. R Development Core Team2.1.; R Foundation for Statistical Computing, Vienna, Austria; 2005) statistical package. One mouse was excluded because of methodic problems.

### Example 4

### Histological Examination

In addition, the lungs of wild-type, lpr, and gld mice were examined histologically for pathognomonic alterations of pneumonitis using standard methods (Penney et al., Int J Radiat Oncol Biol Phys 1994; 29:789-804; Kuwano et al., J Clin Invest 1999; 104:13-9; Travis, Int J Radiat Oncol Biol Phys 1980; 6:345-7). After cervical dislocation, lungs were fixed in formalin and embedded in paraffin. Two five micron-thick sections were cut from each lobe and stained with hematoxillin and eosin. In accordance with previously reported findings (Penney et al., Int J Radiat Oncol Biol Phys 1994; 29:789-804; Kuwano et al., J Clin Invest 1999; 104:13-9; Travis, Int J Radiat Oncol Biol Phys 1980; 6:345-7) lungs were analyzed at day 1, 21, 42 and 84, for the onset of the pneumonitic reaction and at day 210 to detect potential late effects. Histopathological changes, i.e., alveolar wall thickness, interstitial edema, and interstitial and peribronchial inflammation, were judged by two independent investigators (TE, KN) in a blinded manner. For each of these morphologic alterations, a numerical score was determined. Scoring was assessed according to previously published scoring criteria (Penney et al., Int J Radiat Oncol Biol Phys 1994; 29:789-804; Kuwano et al., J Clin Invest 1999; 104:13-9; Travis, Int J Radiat Oncol Biol Phys 1980; 6:345-7) as follows: 0=alterations in less than 10% of the fields viewed, 1=in 10-30%, 2=in 30-50%, 3=in 50-70%, and 4=in more than 70%. A cumulative inflammation score was then determined for each group of mice.

For statistical analysis, a total inflammatory score [Tot Inf] was used, consisting of the sum of the scores for alveolar wall thickness, interstitial edema, and interstitial and peribronchial inflammation. An ordinal logistic fit considering Tot Inf as dependent variable and dose, day, lung (left or right), strain, observer (#1 or #2), dose*strain, dose*day, and dose*observer as model effects (independent variables) was done using the statistical software package JMP (SAS Institute Inc. USA). Since all model effects except “dose*observer” were statistically significant (p<0.05), subgroup analysis was performed excluding the model effect “dose*observer” for each mouse strain, day, and observer resulting in a total number of 27 ordinal logistic fits. To adapt for multiple testing (Bonferroni correction) only model effects reaching P<0.00185 were considered statistically significant.

For histopathological analysis at least five sham-irradiated (0 Gy) and irradiated (12.5 Gy) C57BL6/J, gld, and lpr mice per dose and time point were examined. The exact numbers (N) of left and right lungs used for histopathologic analysis are given below (Tables 4-6).

TABLE 4
Number Left/Right Lungs of C57BL6/J Mice
for Histopathological Examination

| Dose | Day 1 | Day 21 | Day 42 | Day 84 |
|---|---|---|---|---|
| 0 Gy | 7/7 | 7/7 | 7/7 | 7/7 |
| 12.5 Gy | 7/7 | 7/7 | 7/7 | 7/7 |

TABLE 5
Number Left/Right Lungs of gld Mice
for Histopathological Examination

| Dose | Day 1 | Day 21 | Day 42 | Day 84 |
|---|---|---|---|---|
| 0 Gy | 5/5 | 7/7 | 6/7 | 7/7 |
| 12.5 Gy | 6/6 | 6/6 | 5/5 | 7/7 |

TABLE 6
Number Left/Right Lungs of lpr Mice
for Histopathological Examination

| Dose | Day 1 | Day 21 | Day 42 | Day 84 |
|---|---|---|---|---|
| 0 Gy | 5/5 | 5/5 | 5/5 | 5/5 |
| 12.5 Gy | 5/56 | 5/5 | 5/5 | 6/6 |

Due to the development of lymphadenopathy in lpr and gld mice, these analyses were restricted to days 1-84 (Watanabe-Fukunaga et al., Nature 1992; 356:314-7; Takahashi et al., Cell 1994; 76(6):969-76).

### Example 5

### Irradiation Increases Breathing Frequency In Wild Type, But Not In lpr And gld Mice

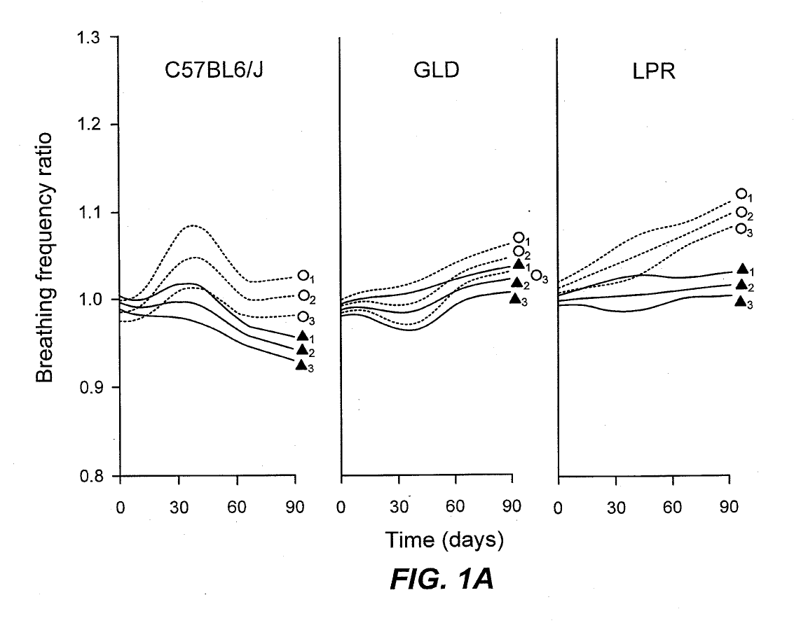

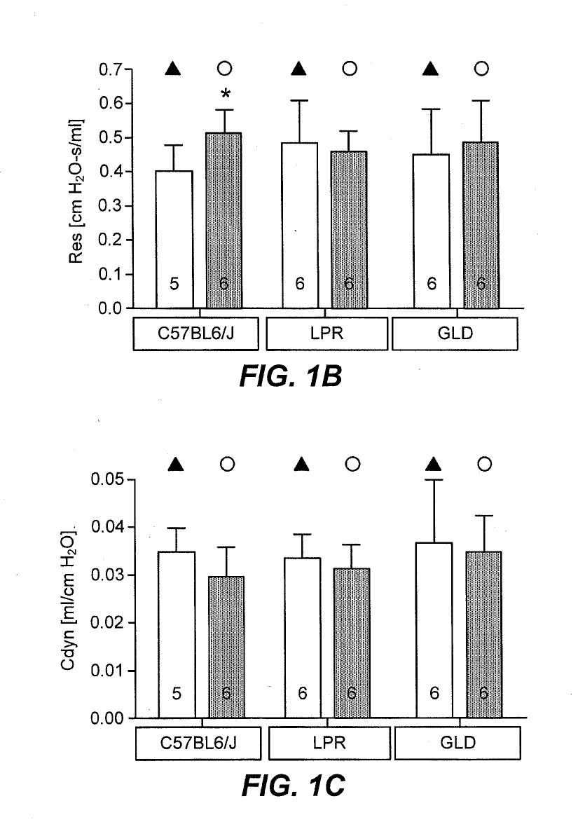

Determination of lung physiologic parameters revealed that in sham-irradiated wild-type mice, breathing frequency ratio decreased over time, whereas that of sham-irradiated lpr and gld mice increased, most probably due to lymphadenopathy e.g., in the thorax, cervical lymph nodes, and lungs, as previously described (Watanabe-Fukunaga et al., Nature 1992; 356:314-7; Takahashi et al., Cell 1994; 76(6):969-76). In contrast to sham-irradiated mice, wild-type mice treated with 12.5 Gy had a statistically significant increase in breathing frequency ratio on days 5-70 (p<0.03) with a maximum at day 37 (1.049±0.035 for 12.5 Gy, p=0.004 versus 0.997±0.021 for 0 Gy, p=0.051 (Mean±95% confidence interval (CI)). (FIG. 1A). This peak in breathing frequency occurred during the acute phase of lung injury (Travis, Radiat Res 1980; 84:133-43) namely pneumonitis. Furthermore, this peak was neither detectable in lpr nor gld mice (day 37: lpr: 1.046±0.023 for 12.5 Gy, p=0.992 versus 1.006±0.020 for 0 Gy, p=0.917; gld: 0.995±0.024 for 12.5 Gy, p=0.086 versus 0.986±0.022 for 0 Gy, p=0.151 (Mean±95% confidence interval (CI)) (FIG. 1A). Although breathing frequency was recorded until day 210, only values through day 90 could be considered for further analysis. From day 90 onward, breathing frequencies of gld and lpr mice increased independent of irradiation, most probably due to lymphadenopathy e.g., in the thorax, the mediastinum, and the bowel, which impairs normal breathing (Watanabe-Fukunaga et al., Nature 1992; 356:314-7; Takahashi et al., Cell 1994; 76(6):969-76).

### Example 6

### Irradiation Increases Airway Resistance In Wild Type, But Not In lpr And gld Mice

Pulmonary resistance and pulmonary compliance were also compared in wild-type, gld, and lpr mice at day 42 after irradiation (FIGS. 1B and C). Irradiated wild-type mice had statistically significant higher airway resistance than sham-irradiated mice at day 42 (0.5115±0.071 for 12.5 Gy versus 0.3974±0.078 for 0 Gy (Mean±95% confidence interval (CI)), p=0.03) (FIG. 1B). Thus, in irradiated wild-type mice, at the time point of maximal increase in breathing frequency, a second physiologic parameter indicative of inflammatory lung injury was increased. In contrast, no differences were observed in irradiated and sham-irradiated gld (p=0.349) and lpr (p=0.349) mice (FIG. 1B).

### Example 7

### Irradiation Leads To Pathognomomic Alterations of Pneumonitis in Wild Type, But Not in lpr And gld Mice

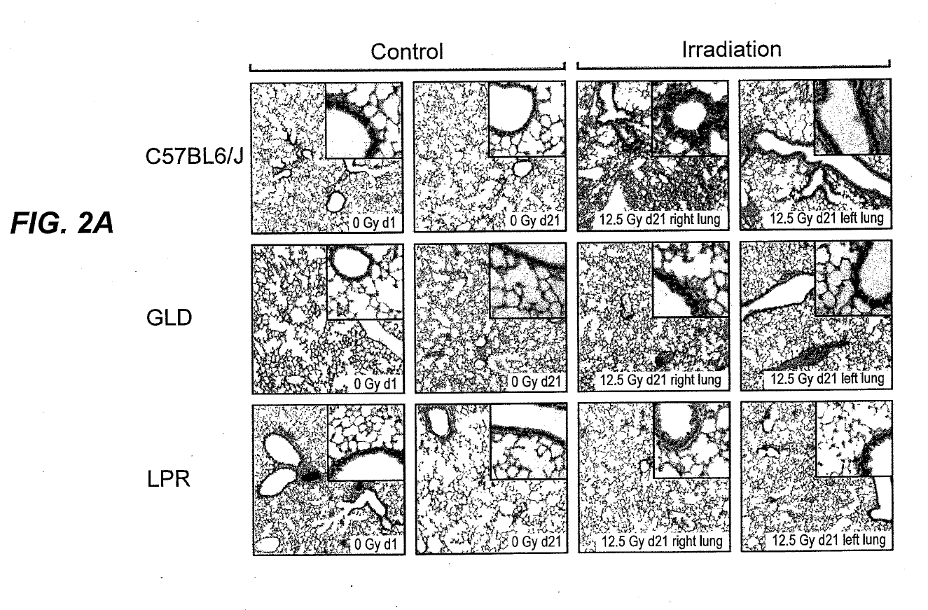

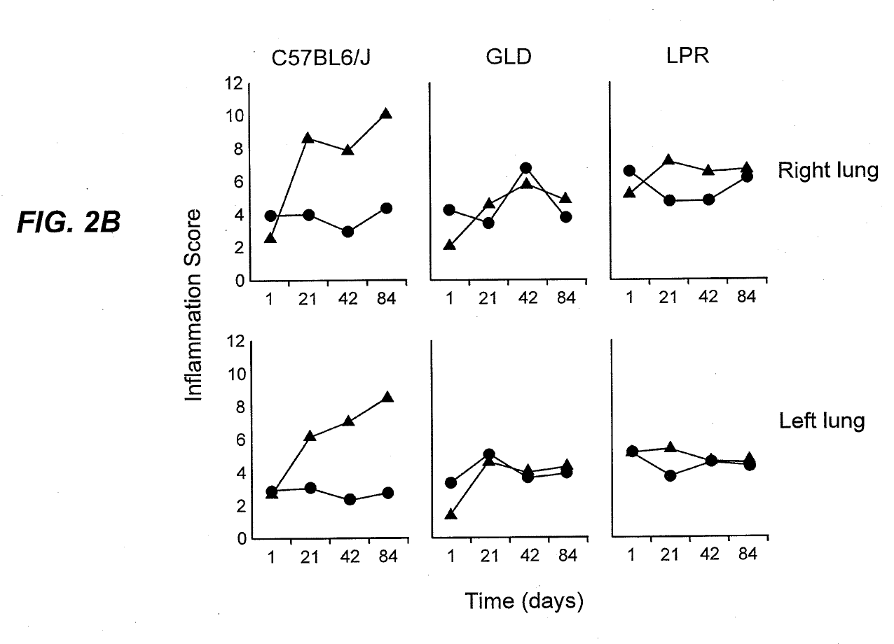

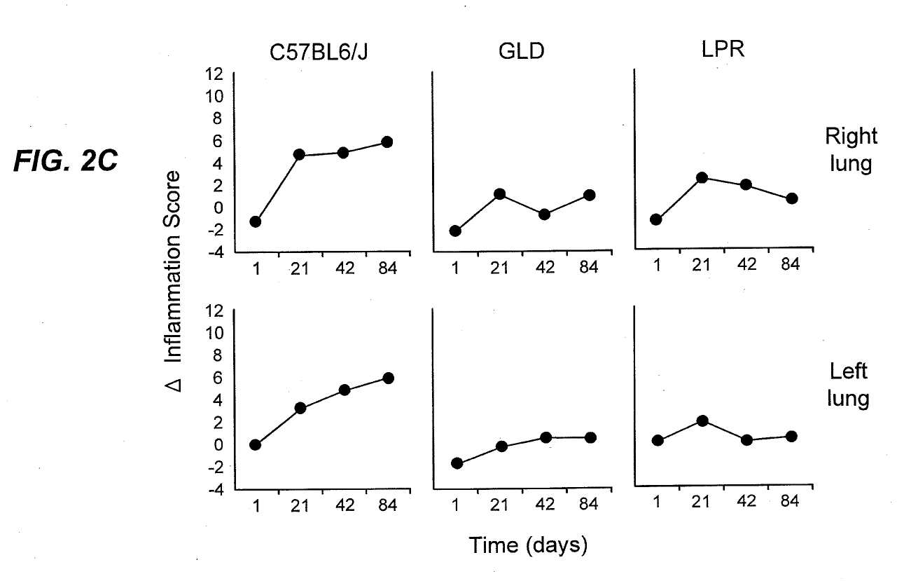

To further confirm the physiological significance of CD95 and CD95L in the development of irradiation induced pneumonitis, the lungs of wild-type, lpr, and gld mice were histologically examined for characteristic pathognomonic alterations of pneumonitis. In sham-irradiated wild-type mice, no morphologic changes in lung tissue were detected throughout the observation period (day 1 to day 84). In contrast, following 12.5 Gy exposure a noteworthy augmentation in alveolar wall thickness, occurrence of interstitial edema, and interstitial and peribronchial inflammation in irradiated right lungs of wild-type mice was detected (FIG. 2A) resulting in increased inflammation scores on days 21, 42, and 84, compared with sham-irradiated mice (FIG. 2B). Moreover, a less pronounced yet clearly detectable inflammatory reaction was observed in the lead-shielded left lungs supporting the interpretation that humoral factors, like inflammatory cytokines or chemokines, might be involved in this form of secondary lung injury (FIGS. 2A and B). In strong contrast to wild-type mice, the histopathological analyses revealed no signs of a pulmonary inflammatory response in gld mice. Irradiated right lungs of gld mice remained undistinguishable from non-irradiated or shielded left lungs at all time points (FIG. 2A).

A slight but statistically non-significant inflammatory response was observed in the right lungs of lpr mice at day 21 and 42 upon 12.5 Gy exposure when compared with sham-irradiated lpr mice (FIGS. 2A and B). However, the difference in the respective inflammation score was clearly less pronounced in lpr mice (˜2 points above non-irradiated littermates) compared with wild-type mice (˜5 points above non-irradiated littermates; P<0.001) (FIG. 2C). Similarly, no statistically significant histological differences were observed in the shielded left lungs of mice treated with 0 Gy and 12.5 Gy (FIGS. 2A and B).

### Example 8

### Effect of CD95-R-Fc Fusion Protein APG101 on the Development of CD95 Mediated-Irradiation Induced Pneumonitis

Despite of intensive irradiation therapy strategies many thorax-associated malignant diseases, such as bronchial carcinoma, have an unfavorable prognosis. The radiation dose required for successful radiation therapy can often not be applied due to insufficient tolerance of the affected lung tissue. Even when radiation dose is restricted, the undesired side effect of lethal irradiation induced pneumonitis often occurs. In various studies (Heinzelmann et al., Journal of the National Cancer Institute, Vol. 98, 1248-51, 2006; Belka et al., Radiation Research, Vol. 149, 588-95, 1998, Neff et al., Am J Pathology, Vol. 166, 685-94) it was reported, that death receptor CD95 and its ligand CD95L are radiation inducible and, in concerting action together with cytokines, chemokines, and adhesion molecules, directly or indirectly affect inflammatory cells. The targeted administration of CD95R-Fc fusion protein comprising the sequence of SEQ ID NO:1 (referred to herein as APG101) will provide a promising approach to prevent the development of irradiation-induced pneumonitis or to cure already manifested diseases. A trial assay may comprise 4 groups (see table 7). Group 1 will receive sham-radiation, group 2 will receive radiation without treatment, group 3 will receive sham-radiation and APG101 (to assess the effect of APG101 alone) and group 4 will receive radiation as well as treatment with APG101. Doses of 1 mg APG101 per animal will be administered intraperitonelly to the treated animal groups 3 and 4 at days −1, 1, 4, 8, 11, 15, 18, 22, 25, 29, 32, 36, 39 and 42. Based on prior experimentation it is calculated that 12 animals per group will be required giving rise to a total number 144 animals per trial. From published data, the extent of the expected differences between the control group (group 1: C57BL6/J mice with sham radiation: 0 Gray (Gy)) compared to radiated controls (group 2: C57BL6/J mice with one-time radiation of 17.5 Gy), the experimental trial groups (group 3: C57BL6/J mice with APG101 administration 1000 μg 2×/week; 0 Gy); (group 4: C57BL6/J mice with APG101 administration 1000 μg 2×/week; 17.5 Gy) and the envisaged end points are known, resulting in a number of 12 animals per group and trial time point.

TABLE 7
Application Schedule APG101 and
Irradiation-Induced Pneumonitis

| | Radiation | | Animal | Necroscopy |
|---|---|---|---|---|
| Group | Dose (Gy) | APG101 | Number | (Animal Number per day) |
| 1 | 0 | No | 36 | 12 at Day 1 |
| | | | | 12 at Day 21 |
| | | | | 12 at Day 42 |
| 2 | 17.5 | No | 36 | 12 at Day 1 |
| | | | | 12 at Day 21 |
| | | | | 12 at Day 42 |
| 3 | 0 | Yes | 36 | 12 at Day 1 |
| | | | | 12 at Day 21 |
| | | | | 12 at Day 42 |
| 4 | 17.5 | Yes | 36 | 12 at Day 1 |
| | | | | 12 at Day 21 |
| | | | | 12 at Day 42 |

During lung examination, each right half of the lungs while left half being blocked of the 4 groups of C57BL6/J mice are irradiated with one one-time radiation (RT) of 0/17.5 Gy with or without intraperitoneal administration of APG101. Sham-irradiation (0 Gy) of mice or APG101 administration with sham-irradiation (0 Gy) is essential to determine potential histopathological alterations of the lung tissue due to aging processes or due to APG101 administration. Examination day 1 (d1), at which no histopathological alterations upon one-time radiation of 0/17.5 Gy are expected, serves as reference status for the histopathological alterations observed after 21/42 days of the trial upon treatments. To measure the time course of developing pneumonitis and the impact of APG101 treatment, animal subgroups (12 animals each) are killed by cervical dislocation after anaesthetizing using carbon dioxide at days 1, 21, and 42, the lungs are removed and studied histopathologically (H&E staining). Histopathological alterations may be expected after 21 or 42 days in group 2 (one-time RT of 17.5 Gy) and no alterations in groups 1 (one-time RT of 0 Gy) and 3 (one-time RT of 0 Gy, APG101 1000 μg 2×/week), and moderate alterations in group 4 (one-time RT of 17.5 Gy, APG101 1000 μg 2×/week).

What is claimed is:

1. A method of minimizing the risk for a patient of developing pneumonitis during radiotherapy for a thorax-associated neoplasm, the method comprising administering to said patient a therapeutically effective amount of a compound which inhibits the CD95/CD95L system in a cell of the patient.

2. The method according to claim 1, wherein the compound is a fusion protein comprising the extracellular domain of CD95.

3. The method according to claim 2, wherein the fusion protein further comprises an oligomerization domain.

4. The method according to claim 2, wherein the fusion protein further comprises an Fc fragment of an IgG immunoglobulin.

5. The method according to claim 4, wherein the fusion protein comprises the sequence of SEQ ID NO:1.

6. The method according to claim 4, wherein the fusion protein consists of the sequence of SEQ ID NO:1.

7. The method according to claim 4, wherein the fusion protein comprises an amino acid sequence which is at least 95% identical to SEQ ID NO:1.

8. The method according to claim 4, wherein the fusion protein comprises an amino acid sequence which is at least 90% identical to SEQ ID NO:1.

9. The method according claim 1, wherein the compound is an antibody against CD95 or CD95L.

10. A method of increasing the radiation dose administered to a patient during radiotherapy for a thorax-associated neoplasm, the method comprising administering to said patient a therapeutically effective amount of a compound which inhibits the CD95/CD95L system in a cell of the patient.

11. The method according to claim 10, wherein the radiation dose can be increased by at least 10%.

12. The method according to claim 10, wherein the radiation dose can be increased by at least 15%.

13. The method according to claim 10, wherein the radiation dose can be increased by at least 10 to 15%.

14. The method according to claim 10, wherein the compound is a fusion protein comprising the extracellular domain of CD95.

15. The method according to claim 14, wherein the fusion protein further comprises an Fc fragment of an IgG immunoglobulin.

16. The method according to claim 15, wherein the fusion protein comprises the sequence of SEQ ID NO:1.

17. The method according to claim 15, wherein the fusion protein consists of the sequence of SEQ ID NO:1.

18. The method according to claim 15, wherein the fusion protein comprises an amino acid sequence which is at least 95% identical to SEQ ID NO:1.

19. A method for identifying a compound for the treatment or prevention of the development of radiation-induced pneumonitis comprising the steps of:
(a) contacting a peptide derived from the CD95/CD95L system to a test substance under conditions allowing binding of the test substance to the peptide, and
(b) determining whether the test substance inhibits an activity of the peptide.

20. The method according to claim 19, wherein step (b) is performed by using an in-vitro cell system capable of mimicking the cellular conditions occurring with radiation-induced pneumonitis.


SEQUENCE LISTING (1 sequence)

SEQ ID NO: 1 - protein, 400 aa, Artificial Sequence
  CD95R-Fc fusion protein, APG101, CD95/CD95 inhibitor, fusion protein of extracellular domain of death receptor CD95 and Fc fragment of IgG immunoglobulin
MVGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK